(19) United States
(12) Patent Application Publication
(10) Pub. No.: US 2026/0108450 A1
(43) Pub. Date: Apr. 23, 2026

(54) COSMETIC COMPOSITION FOR ENHANCING SKIN ABSORPTION AND FORMULATION STABILITY

(71) Applicant: CELLTEM BIO INC., Seoul (KR)

(72) Inventors: Eun Hae CHO, Seoul (KR); Ji Young LEE, Gwangmyeong-si (KR); Hyo Won JEON, Seoul (KR); Jin Ha PARK, Yongin-si (KR); Eun Aa KO, Seoul (KR); Na Yeon KIM, Seoul (KR); Hyeon Ji KIM, Yongin-si (KR)

(73) Assignee: CELLTEM BIO INC., Seoul (KR)

(21) Appl. No.: 19/118,069

(22) PCT Filed: Jan. 4, 2023
(86) PCT No.: PCT/KR2023/000139

§ 371 Date: Apr. 3, 2025

(30) Foreign Application Priority Data
Oct. 5, 2022 (KR) 10-2022-0126895

(51) Int. Cl. A61K 8/67; A61K 8/34; A61K 8/37; A61K 8/49; A61K 8/55; A61K 8/86; A61K 8/92; A61Q 19/00

(52) U.S. Cl. CPC A61K 8/675; A61K 8/34; A61K 8/347; A61K 8/375; A61K 8/4946; A61K 8/4953; A61K 8/498; A61K 8/553; A61K 8/678; A61K 8/86; A61K 8/922; A61Q 19/00; A61K 2800/48

(57) ABSTRACT

A cosmetic composition for enhancing skin absorption of active ingredients and formulation stability is disclosed. The cosmetic composition contains three or more ingredients selected from the group consisting of bis-ethoxydiglycolcyclohexane 1,4-dicarboxylate, lysolecithin, citron seed oil, and glycereth-25 PCA isostearate. The cosmetic composition has effects of enhancing skin absorption of active ingredients and enhancing formulation stability.

### TECHNICAL FIELD

The present invention relates to a cosmetic composition for enhancing skin absorption of active ingredients and formulation stability.

### BACKGROUND ART

In general, the skin is composed of the stratum corneum, epidermis, and dermis, and functions to protect against external environmental stimuli. The skin is the organ with the largest surface area in the human body and may effectively deliver beneficial ingredients such as drugs when an appropriate method is used. However, because of the stratum corneum, which is the outermost layer of the skin, the skin permeability of external substances is extremely low, making it difficult to deliver substances. In order to overcome the defense mechanism of the skin barrier against external substances when external substances are delivered to the skin, research on skin penetration methods has been attempted, but it is considered almost impossible to deliver drugs through the properties of external substances without physically damaging or irritating the skin.

In the current cosmetics market, there is a need for various methods of promoting transdermal permeability. One approach is blending specific ingredients that promote transdermal permeability and another is utilizing formulations such as liposome compositions.

### DISCLOSURE

### Technical Problem

In the present invention, it was confirmed that the permeability and formulation stability of active ingredients differ depending on the type of thickener constituting the cosmetic composition, the type of transdermal permeability enhancer or solubilizer, or a combination of transdermal permeability enhancers, and the optimal combination thereof was identified to develop an optimal composition that can increase the skin permeation/absorption of active ingredients and enhance formulation stability.

### Technical Solution

In the present invention, a cosmetic composition which maintains a stable state although bis-ethoxydiglycol cyclohexane 1,4-dicarboxylate and lysolecithin, which are transdermal permeability enhancers, are applied, was developed, and research was conducted to optimize the transdermal permeation of active (efficacious) ingredients contained in the cosmetic composition using an additional transdermal permeability enhancer in addition to the two transdermal permeability enhancers described above. In addition, in order to develop a cosmetic composition which remains stable although bis-ethoxydiglycol cyclohexane 1,4-dicarboxylate and lysolecithin are applied, a cosmetic composition with enhanced skin absorption and formulation stability was developed using an optimal thickener combination and glycereth-25 PCA isostearate or citron seed oil as a skin penetration enhancer. Based on this finding, the present invention was completed.

Accordingly, it is one object of the present invention to provide a cosmetic composition for enhancing skin absorption of an active ingredient and formulation stability, containing at least three ingredients selected from the group consisting of bis-ethoxydiglycol cyclohexane 1,4-dicarboxylate, lysolecithin, citron seed oil, and glycereth-25 PCA isostearate.

It is another object of the present invention to provide use of at least three ingredients selected from the group consisting of bis-ethoxydiglycol cyclohexane 1,4-dicarboxylate, lysolecithin, citron seed oil, and glycereth-25 PCA isostearate for preparation of a cosmetic composition for enhancing skin absorption of an active ingredient and formulation stability.

It is another object of the present invention to provide use of at least three ingredients selected from the group consisting of bis-ethoxydiglycol cyclohexane 1,4-dicarboxylate, lysolecithin, citron seed oil, and glycereth-25 PCA isostearate for enhancement of skin absorption of an active ingredient and formulation stability.

It is another object of the present invention to provide at least three ingredients selected from the group consisting of bis-ethoxydiglycol cyclohexane 1,4-dicarboxylate, lysolecithin, citron seed oil, and glycereth-25 PCA isostearate for enhancement of skin absorption of an active ingredient and formulation stability.

It is another object of the present invention to provide a method of enhancing skin absorption of an active ingredient and formulation stability including applying, to the skin of a subject, a cosmetic composition containing at least three ingredients selected from the group consisting of bis-ethoxydiglycol cyclohexane 1,4-dicarboxylate, lysolecithin, citron seed oil, and glycereth-25 PCA isostearate, and/or the active ingredient.

It is another object of the present invention to provide a method of enhancing skin absorption of an active ingredient and formulation stability including applying, to the skin of a subject, at least three ingredients selected from the group consisting of bis-ethoxydiglycol cyclohexane 1,4-dicarboxylate, lysolecithin, citron seed oil, and glycereth-25 PCA isostearate, and/or the active ingredient.

### Advantageous Effects

The present invention relates to a cosmetic composition for enhancing skin absorption of an active ingredient and formulation stability, containing at least three ingredients selected from the group consisting of bis-ethoxydiglycol cyclohexane 1,4-dicarboxylate, lysolecithin, citron seed oil, and glycereth-25 PCA isostearate.

The cosmetic composition of the present invention has the effect of enhancing the skin absorption of active ingredients and the formulation stability.

### BEST MODE

Hereinafter, the present invention will be described in more detail.

Meanwhile, descriptions and embodiments provided herein may also be applied to other descriptions and embodiments. That is, all combinations of various elements disclosed herein fall within the scope of the present invention. In addition, the following specific description should not be construed as limiting the scope of the present invention.

Terms such as “comprising” used herein should be understood as being open-ended terms that have the possibility of including another element or embodiment, unless specifically stated otherwise in the phrase or sentence containing the term.

Terms or words used in the description and claims according to the present invention should not be construed as limited to ordinary or dictionary terms, and should be construed with meanings and concepts that are consistent with the technical idea of the present disclosure based on the principle that the inventors may appropriately define concepts of the terms to appropriately describe their own disclosure in the best way.

In one aspect, the present invention is directed to a cosmetic composition for enhancing skin absorption of an active ingredient and formulation stability, containing at least three ingredients selected from the group consisting of bis-ethoxydiglycol cyclohexane 1,4-dicarboxylate, lysolecithin, citron seed oil, and glycereth-25 PCA isostearate.

The bis-ethoxydiglycol cyclohexane 1,4-dicarboxylate, lysolecithin, citron seed oil, and glycereth-25 PCA isostearate according to the present invention act as transdermal permeability enhancers to promote skin absorption of the active ingredient.

In the present invention, the active ingredient may be contained in an amount of 1.5 to 10% (w/w) based on 100% (w/w) of the cosmetic composition and may be selected from the group consisting of niacinamide, allantoin, bakuchiol, panthenol, resveratrol, ectoine, alpha-bisabolol, glabridin, rucinol, and tocopherol, but is not limited thereto.

In one embodiment of the present invention, the cosmetic composition of the present invention contains bis-ethoxydiglycol cyclohexane 1,4-dicarboxylate, lysolecithin and citron seed oil, which contribute to enhancing skin absorption as transdermal permeability enhancers, which function to improve skin absorption of at least one selected from the group consisting of niacinamide, allantoin and bakuchiol as an active ingredient.

The cosmetic composition for enhancing skin absorption of niacinamide, allantoin or bakuchiol contains 0.3 to 2.0% (w/w) of bis-ethoxydiglycol cyclohexane 1,4-dicarboxylate, 0.01 to 0.15% (w/w) of lysolecithin, and 0.5 to 7.0% (w/w) of the citron seed oil, which function as transdermal permeability enhancers.

More specifically, the cosmetic composition for enhancing skin absorption of niacinamide, allantoin or bakuchiol contains 0.4 to 0.6% (w/w) of bis-ethoxydiglycol cyclohexane 1,4-dicarboxylate, 0.05 to 0.10% (w/w) of lysolecithin and 1.0 to 5.0% (w/w) of citron seed oil.

In addition, the cosmetic composition may contain diutan gum as a thickener. When only diutan gum is used as a thickener instead of ammonium polyacryloyldimethyl taurate, the transdermal permeability increases, but phase separation occurs. When a combination of diutan gum and ammonium polyacryloyldimethyl taurate is used as a thickener, the phase separation may be suppressed and the formulation stability may be improved. However, since ammonium polyacryloyldimethyl taurate improves the formulation stability but slightly lowers the transdermal permeability. Therefore, when citron seed oil is used in combination with bis-ethoxydiglycol cyclohexane 1,4-dicarboxylate and lysolecithin, the effect of improving the transdermal permeability of the active ingredients (niacinamide, allantoin, bakuchiol) may be obtained.

In one embodiment of the present invention, the cosmetic composition of the present invention contains bis-ethoxydiglycol cyclohexane 1,4-dicarboxylate, lysolecithin and glycereth-25 PCA isostearate as transdermal permeability enhancers, which contribute to enhancing skin absorption, and thus function to enhance skin absorption of at least one selected from the group consisting of niacinamide, panthenol, resveratrol, ectoine, glabridin, alpha-bisabolol, rucinol, and bakuchiol as an active ingredient.

The cosmetic composition for enhancing skin absorption of niacinamide, panthenol or resveratrol contains 0.3 to 2.0% (w/w) of bis-ethoxydiglycol cyclohexane 1,4-dicarboxylate, 0.01 to 1.0% (w/w) of lysolecithin and 0.1 to 2.0% (w/w) of glycereth-25 PCA isostearate, which function as transdermal permeability enhancers.

More specifically, the cosmetic composition for enhancing skin absorption of niacinamide, panthenol or resveratrol contains 0.4 to 0.6% (w/w) of bis-ethoxydiglycol cyclohexane 1,4-dicarboxylate, 0.015 5 to 0.5% (w/w) of lysolecithin, and 0.3 to 1.5% (w/w) of glycereth-25 PCA isostearate.

In addition, the cosmetic composition containing the niacinamide, panthenol or resveratrol as an active ingredient may contain glycereth-25 PCA isostearate as a solubilizing agent. When glycereth-25 PCA isostearate is used as an ingredient constituting a soluble moiety (soluble phase) instead of the PEG-40 hydrogenated castor oil, there is an effect of increasing transdermal permeability. Therefore, glycereth-25 PCA isostearate may be used as a transdermal permeability enhancer instead of PEG-40 hydrogenated castor oil.

In one embodiment of the present invention, the cosmetic composition for enhancing skin absorption of niacinamide, ectoine or resveratrol contains 0.3 to 2.0% (w/w) of bis-ethoxydiglycol cyclohexane 1,4-dicarboxylate, 0.01 to 1.0% (w/w) of lysolecithin, and 0.1 to 2.0% (w/w) of glycereth-25 PCA isostearate, which function as transdermal permeability enhancers.

More specifically, the cosmetic composition for enhancing skin absorption of niacinamide, ectoine or resveratrol contains 0.4 to 2.0% (w/w) of bis-ethoxydiglycol cyclohexane 1,4-dicarboxylate, 0.015 to 0.5% (w/w) of lysolecithin, and 0.3 to 1.5% (w/w) of glycereth-25 PCA isostearate.

In addition, the cosmetic composition containing the niacinamide, panthenol or resveratrol as active ingredients may contain diutan gum, xanthan gum, carbomer, or a combination of xanthan gum and carbomer as a thickener. Xanthan gum or carbomer used as the thickener may exhibit excellent transdermal permeation rate and improved formulation transparency, as compared to diutan gum.

In one embodiment of the present invention, the cosmetic composition of the present invention contains bis-ethoxydiglycol cyclohexane 1,4-dicarboxylate, lysolecithin, citron seed oil, and glycereth-25 PCA isostearate as transdermal permeation enhancers, which contribute to enhancing skin absorption, and function to enhance skin absorption of at least one selected from the group consisting of niacinamide, alpha-bisabolol, glabridin and ectoine as an active ingredient.

The cosmetic composition for enhancing skin absorption of niacinamide, alpha-bisabolol, or glabridin contains 0.3 to 2.0% (w/w) of bis-ethoxydiglycol cyclohexane 1,4-dicarboxylate, 0.01 to 1.0% (w/w) of lysolecithin, 0.5 to 5.0% (w/w) of citron seed oil, and 0.1 to 2.0% (w/w) of glycereth-25 PCA isostearate, which function as transdermal permeability enhancers.

More specifically, the cosmetic composition for enhancing skin absorption of niacinamide, alpha-bisabolol, or glabridin contains 0.4 to 1.2% (w/w) of bis-ethoxydiglycol cyclohexane 1,4-dicarboxylate, 0.015 to 0.5% (w/w) of lysolecithin, 1.0 to 3.0% (w/w) of citron seed oil, and 0.3 to 1.5% (w/w) of glycereth-25 PCA isostearate.

The cosmetic composition for enhancing skin absorption of niacinamide, alpha-bisabolol or glabridin caused phase separation when the carbomer and xanthan gum as thickeners were used in combination with bis-ethoxydiglycol cyclohexane 1,4-dicarboxylate (Comparative Example 8). In Comparative Example 9, in which ammonium polyacryloyldimethyl taurate was used as a thickener in addition to carbomer and xanthan gum, there was no phase separation, but the transdermal permeability of the active ingredient was significantly reduced. In Examples 13 and 14 in which acrylate/C10-30 alkyl acrylate crosspolymer was used in addition to carbomer and xanthan gum, there was no phase separation and good transdermal permeability of the active ingredient was observed.

Therefore, the cosmetic composition for enhancing the skin absorption of niacinamide, alpha-bisabolol or glabridin has the effect of enhancing transdermal permeability without causing phase separation when carbomer, xanthan gum and acrylate/C10-30 alkyl acrylate crosspolymer are used in combination as thickeners.

The cosmetic composition has no particular limitation as to the formulation thereof and may be used on the skin, mucous membrane, scalp or hair, and may be formulated as a solution, suspension, emulsion, paste, gel, cream, lotion, powder, soap, surfactant-containing cleansing, oil, powder foundation, emulsion foundation, wax foundation spray or the like. More specifically, the cosmetic composition may be formulated into a skin cosmetic composition such as a softening toner, a nourishing toner, a lotion, a cream, a pack, a gel, a patch, an oil-in-water (O/W) type formulation, a water-in-oil (O/W) type formulation, a solubilizer, a color cosmetic composition such as a lipstick, a makeup base or a foundation, a cleansing composition such as a shampoo, a rinse, a body cleanser, a toothpaste or a mouthwash, or a hair cosmetic composition such as a hair styling agent, for example, a hair tonic, a hair gel or a hair mousse, a hair treatment or a hair dye.

In addition, appropriate ingredients in the cosmetic composition of each formulation may be selected and mixed depending on the formulation or purpose of use of the cosmetic.

The cosmetic composition of the present invention may contain appropriate additives as needed, and may, for example, further contain ingredients such as a preservative, a pigment, and an additive commonly used in the art.

In another aspect, the present invention is directed to use of at least three ingredients selected from the group consisting of bis-ethoxydiglycol cyclohexane 1,4-dicarboxylate, lysolecithin, citron seed oil, and glycereth-25 PCA isostearate for preparation of a cosmetic composition for enhancing skin absorption of an active ingredient and formulation stability.

In another aspect, the present invention is directed to use of at least three ingredients selected from the group consisting of bis-ethoxydiglycol cyclohexane 1,4-dicarboxylate, lysolecithin, citron seed oil, and glycereth-25 PCA isostearate for enhancement of skin absorption of an active ingredient and formulation stability.

In another aspect, the present invention is directed to at least three ingredients selected from the group consisting of bis-ethoxydiglycol cyclohexane 1,4-dicarboxylate, lysolecithin, citron seed oil, and glycereth-25 PCA isostearate for enhancement of skin absorption of an active ingredient and formulation stability.

In another aspect, the present invention is directed to a method of enhancing skin absorption of an active ingredient and formulation stability including applying, to the skin of a subject, a cosmetic composition containing at least three ingredients selected from the group consisting of bis-ethoxydiglycol cyclohexane 1,4-dicarboxylate, lysolecithin, citron seed oil, and glycereth-25 PCA isostearate, and/or the active ingredient.

In another aspect, the present invention is directed to a method of enhancing skin absorption of an active ingredient and formulation stability including applying, to the skin of a subject, at least three ingredients selected from the group consisting of bis-ethoxydiglycol cyclohexane 1,4-dicarboxylate, lysolecithin, citron seed oil, and glycereth-25 PCA isostearate, and/or the active ingredient.

In the present invention, the terms “bis-ethoxydiglycol cyclohexane 1,4-dicarboxylate”, “lysolecithin”, “citron seed oil”, “glycereth-25 PCA isostearate”, “cosmetic composition”, “active ingredient”, “skin absorption”, and “formulation stability” are as described above.

The subjects to whom the cosmetic composition of the present invention or at least three ingredients and/or the active ingredient is applied include mammals including human without limitation, but may be, for example, cows, pigs, horses, rabbits, mice, and humans.

The method of enhancing skin absorption of the active ingredient and formulation stability according to the present invention includes administering to the skin of a subject at least three ingredients selected from the group consisting of bis-ethoxydiglycol cyclohexane 1,4-dicarboxylate, lysolecithin, citron seed oil, and glycereth-25 PCA isostearate, or a cosmetic composition containing the same.

As used herein, the term “application” means bringing the three or more ingredients or the cosmetic composition containing the same and the active ingredient into contact with the skin of a subject by any appropriate method, and includes, but is not limited to, all operations to allow the at least three ingredients or the cosmetic composition and the active ingredient to be absorbed by the target skin tissue.

### MODE FOR INVENTION

Hereinafter, embodiments of the present invention will be described in detail with reference to the accompanying drawings so that the present invention may be implemented by those skilled in the art. However, the present invention may be embodied in many different forms and is not limited to the embodiments described herein.

### Experimental Example 1. Preparation of Emulsifying Composition Using Active Ingredients (Niacinamide, Allantoin, and Bakuchiol)

### 1-1. Preparation of Emulsifying Compositions of Comparative Examples 1 to 3

The emulsifying compositions of Comparative Examples 1 to 3 were prepared according to the compositions set forth in Table 1. The ingredients constituting an aqueous phase in Table 1 were injected into a dissolving tank, heated (70 to 75° C.) and agitated to form the aqueous phase. Then, the ingredients constituting an oil phase were injected into a separate dissolving tank, heated (70 to 75° C.) and agitated to form the oil phase. The oil phase was added to the aqueous phase and homogeneously emulsified at 5,000 rpm for 3 minutes to form an emulsion (emulsifying composition) which was allowed to cool to room temperature. The emulsifying composition was mixed with a thickener and active ingredients (useful ingredients) of niacinamide, allantoin, and bakuchiol, followed by stirring, to prepare a cosmetic composition containing useful ingredients.

EMULFREE® CBG MB, used as an oil ingredient, is a gelling agent containing stearyl alcohol, butylene glycol cocoate, and ethyl cellulose, and has high spreadability because it can relatively unstably capture an oil with a Bi-gel (double gel) system, but has poor stability against phase separation. Therefore, selection of a thickener is very important.

The emulsifying compositions of Comparative Examples 1 to 3 are prepared only using different thickeners. Diutan gum was used as a thickener in Comparative Example 1, ammonium polyacryloyldimethyl taurate was used as a thickener in Comparative Example 2, and polyurethane-15 was used as a thickener in Comparative Example 3.

Emulsifying compositions of Comparative Examples 1 to 3 were prepared using diutan gum, which had been found to have good EGF permeability in an EGF ampoule formulation developed through comparative experiments by the present inventors, and using ammonium polyacryloyldimethyl taurate and polyurethane-15 raw materials which were advantageous for stabilizing emulsions due to the emulsifying power thereof.

The ingredient of the prepared emulsifying composition is expressed based on 100% (w/w).

TABLE 1

| | | Comparative | Comparative | Comparative |
|---|---|---|---|---|
| Phase | Ingredient name | Example 1 | Example 2 | Example 3 |
| Aqueous | Purified water | to 100 | to 100 | to 100 |
| phase | 2,3-Butanediol | 20.00 | 20.00 | 20.00 |
| | Lysolecithin | 0.02 | 0.02 | 0.02 |
| | Bis-ethoxydiglycol cyclohexane1,4- | 2.00 | 2.00 | 2.00 |
| | dicarboxylate | | | |
| | Niacinamide | 4.00 | 4.00 | 4.00 |
| | Allantoin | 0.20 | 0.20 | 0.20 |
| | Adenosine | 0.04 | 0.04 | 0.04 |
| | Ammonium glycyrrhizate | 0.50 | 0.50 | 0.50 |
| Thickener | Diutan gum (Sphingomonas fermented extract) | 0.30 | — | — |
| | Ammonium polyacryloyldimethyl taurate | — | 0.30 | — |
| | polyurethane-15 | — | — | 2.00 |
| Oil phase | EMULFREE ® CBG MB (stearyl alcohol, | 1.00 | 1.00 | 1.00 |
| | butylene glycol cocoate, ethyl cellulose) | | | |
| | Caprylic/capric triglyceride | 2.00 | 2.00 | 2.00 |
| | Dicaprylyl carbonate | 2.00 | 2.00 | 2.00 |
| | Bakuchiol | 0.50 | 0.50 | 0.50 |

### 1-2. Analysis of Transdermal Permeability of Comparative Examples 1 to 3

### 1) Transdermal Permeability Test

The skin absorption rate was evaluated as follows. The cosmetic compositions prepared in Comparative Examples 1 to 3 were applied to the skin. After 8 hours, the transdermal cumulative absorption amount (ppm) of the active ingredient to be analyzed was analyzed by HPLC. At this time, the transdermal cumulative absorption amount (ppm) was evaluated using a Franz diffusion cell (effective area: 1.76625 cm2, volume of receptor chamber: 16 ml), and the skin absorption/permeability was evaluated based thereon.

Specifically, the prepared epidermal layer (2 cm×2 cm) of human skin was prepared such that the stratum corneum was positioned on the receptor chamber, and covered with the donor chamber and fixed with a clamp. Then, the Franz diffusion cell was filled with the receptor phase and maintained at 32.5±0.5° C. The rotation speed (rpm) of the stirrer was maintained at 600±10 so that the permeated test substance was homogeneously mixed. Then, 200 μg of the cosmetic compositions prepared in Comparative Examples 1 to 3 was homogeneously applied to the donor area. At this time, 50% ethanol was used as the receptor phase. The cosmetic composition was applied to the donor area. After 4 and 8 hours, 400 μl of the receptor phase was collected and analyzed by HPLC, and the amount of the collected receptor phase was supplemented with a fresh 50% ethanol solution.

The skin was commercially available from Biohead. The skin was stored at about −20° C., thawed at room temperature for 20 minutes and then used in the experiment.

### 2) Results of Transdermal Permeability Analysis

The cosmetic compositions of Comparative Examples 1 to 3 were applied to the skin. After 8 hours, transdermal permeability was measured and the results are shown in Table 2 below.

In Comparative Examples 1 to 3, only the thickener was changed. Diutan gum was used as the thickener for Comparative Example 1, ammonium polyacryloyldimethyl taurate was used as the thickener for Comparative Example 2, and polyurethane-15 was used as the thickener for Comparative Example 3.

The transdermal permeability of niacinamide, allantoin, and bakuchiol contained in each composition was comparably analyzed based on the 8-hour permeation results. As a result, the transdermal permeability of Comparative Example 1, in which diutan gum was used as the thickener, was the best, and the transdermal permeability of Comparative Examples 2 and 3 was significantly lower. Unlike diutan gum, ammonium polyacryloyldimethyl taurate and polyurethane-15 reduced transdermal permeability, and in particular, polyurethane-15 significantly reduced transdermal permeability. Therefore, polyurethane-15 was excluded to prepare the emulsifying composition.

However, Comparative Example 1 exhibited excellent transdermal permeability of useful ingredients (niacinamide, allantoin, bakuchiol), but caused phase separation in which the oil phase (oil phase) floated to the top in terms of long-term stability. Therefore, an experiment was conducted to develop an emulsifying composition that is capable of improving the permeability while using an appropriate amount of ammonium polyacryloyldimethyl taurate that can avoid the phase separation.

TABLE 2

| Transdermal | Comparative | Comparative | Comparative |
|---|---|---|---|
| penetration (ppm) | Example 1 | Example 2 | Example 3 |
| Niacinamide | 17.18 | 11.71 | 2.5 |
| Allantoin | 1.72 | 0.22 | Not detected |
| Bakuchiol | 0.44 | 0.22 | Not detected |

### Experimental Example 2. Preparation of Emulsifying Compositions Using Active Ingredients (Niacinamide, Allantoin, Bakuchiol)

### 2-1. Preparation of Emulsifying Compositions of Comparative Examples 4 to 6 and Examples 1 to 3

The emulsifying compositions of Comparative Examples 4 to 6 and Examples 1 to 3 are prepared according to the compositions shown in Table 3, and were prepared in the same manner as in the emulsifying compositions of Comparative Examples 1 to 3.

TABLE 3

| | | Comparative | Comparative | Comparative | Example | Example | Example |
|---|---|---|---|---|---|---|---|
| Phase | Ingredient name | Example 4 | Example 5 | Example 6 | 1 | 2 | 3 |
| Aqueous | Purified water | to 100 | to 100 | to 100 | to 100 | to 100 | to 100 |
| phase | 2,3-Butanediol | 20.00 | 20.00 | 20.00 | 20.00 | 20.00 | 20.00 |
| | Lysolecithin | 0.02 | 0.02 | 0.02 | 0.02 | 0.02 | 0.02 |
| | Bis-ethoxydiglycol cyclohexane | 2.00 | 1.00 | 0.50 | 0.50 | 0.50 | 0.50 |
| | 1,4-dicarboxylate | | | | | | |
| | Niacinamide | 4.00 | 4.00 | 4.00 | 4.00 | 4.00 | 4.00 |
| | Allantoin | 0.20 | 0.20 | 0.20 | 0.20 | 0.20 | 0.20 |
| | Adenosine | 0.04 | 0.04 | 0.04 | 0.04 | 0.04 | 0.04 |
| | Ammonium glycyrrhizate | 0.50 | 0.50 | 0.50 | 0.50 | 0.50 | 0.50 |
| Thickener | Diutan gum (Sphingomonas | 0.20 | 0.20 | 0.20 | 0.20 | 0.20 | 0.20 |
| | fermented extract) | | | | | | |
| | Ammonium polyacryloyldimethyl | 0.10 | 0.10 | 0.10 | 0.10 | 0.10 | 0.10 |
| | taurate | | | | | | |
| Oil | EMULFREE ® CBG MB(stearyl | 1.00 | 1.00 | 1.00 | 1.00 | 1.00 | 1.00 |
| phase | alcohol, butylene glycol cocoate, | | | | | | |
| | ethyl cellulose) | | | | | | |
| | Caprylic/capric triglyceride | 2.00 | 2.00 | 2.00 | 1.00 | — | — |
| | Citron seed oil | — | — | — | 1.00 | 2.00 | 5.00 |
| | Dicaprylyl carbonate | 2.00 | 2.00 | 2.00 | 2.00 | 2.00 | — |
| | Bakuchiol | 0.50 | 0.50 | 0.50 | 0.50 | 0.50 | 0.50 |

### 2-2. Formulation Analysis of Comparative Examples 4 to 6 and Examples 1 to 3 (Transdermal Permeability and Stability Against Phase Separation)

The stability against phase separation and transdermal permeability were analyzed using the emulsifying compositions of Comparative Examples 4 to 6 and the emulsifying compositions of Examples 1 to 3 prepared above.

The thickener, ammonium polyacryloyldimethyl taurate, used in Comparative Examples 2 and 3, reduced the transdermal permeability of the active ingredient in the composition, but was incorporated in an amount of 0.1% so as to stabilize the phase separation of the formulation.

In Comparative Examples 4 and 5, in which the emulsifying compositions were prepared using 0.10% of the thickening agent (ammonium polyacryloyldimethyl taurate) and 2.0% or 1.0% of the transdermal permeability enhancer (bis-ethoxydiglycol cyclohexane 1,4-dicarboxylate), the thickening power was weakened and phase separation was observed.

Therefore, the emulsifying compositions of Comparative Example 6 and Examples 1 to 3 were prepared by lowering the amount of ammonium polyacryloyldimethyl taurate to 0.1% and bis-ethoxydiglycol cyclohexane 1,4-dicarboxylate to 0.5%, and the formulation properties of the emulsifying compositions of Comparative Example 6 and Examples 1 to 3 were stabilized. However, since the permeation-blocking polymer (ammonium polyacryloyldimethyl taurate) that was found to have a transdermal permeation-blocking effect in Comparative Example 2 was added and the content of the permeation enhancer (bis-ethoxydiglycol cyclohexane 1,4-dicarboxylate) was lowered, the transdermal permeation of the useful ingredients in Comparative Example 6 decreased.

Therefore, in order to increase the transdermal permeability of the active ingredients, citron seed oil was further added as an oil phase ingredient in Examples 1 to 3. Citron seed oil is known to contain more than 60% of unsaturated fatty acids such as oleic acid, linoleic acid, and linolenic acid, and in particular, more than 30% of each of oleic acid and linoleic acid. Examples 1 to 3 correspond to emulsifying compositions in which citron seed oil was further used at 1%, 2%, and 5%, respectively, compared to Comparative Example 6. The addition of citron seed oil improved the transdermal permeability of useful ingredients (niacinamide, allantoin, bakuchiol), and in particular, the transdermal permeability improvement of useful ingredients such as bakuchiol increased by up to 386% in Example 3 compared to Comparative Example 6.

TABLE 4

| | | Comparative | Comparative | Comparative | Example | Example | Example |
|---|---|---|---|---|---|---|---|
| Formulation analysis | | Example 4 | Example 5 | Example 6 | 1 | 2 | 3 |
| Properties | Phase | Phase | Phase | Stable | Stable | Stable | Stable |
| | separation | separation | separation | | | | |
| | stability | | | | | | |
| | confirmed | | | | | | |
| | for 1 month | | | | | | |
| | at 50° C. | | | | | | |
| Transdermal | Niacinamide | — | — | 11.21 | 11.43 | 15.78 | 17.84 |
| penetration(ppm) | Allantoin | — | — | 0.87 | 0.84 | 0.93 | 1.14 |
| | Bakuchiol | — | — | 0.43 | 0.58 | 0.92 | 1.66 |

### Experimental Example 3. Preparation of Emulsifying Composition Using Active Ingredients (Niacinamide, Allantoin, Bakuchiol)

### 3-1. Preparation of Emulsifying Compositions of Examples 4 to 5

The emulsifying compositions of Examples 4 to 5 are prepared according to the compositions set forth in Table 5 in the same manner as in the emulsifying compositions of Comparative Examples 1 to 3.

TABLE 5

| Phase | Ingredient name | Example 4 | Example 5 |
|---|---|---|---|
| Aqueous | Purified water | to 100 | to 100 |
| phase | 2,3-Butanediol | 20.00 | 20.00 |
| | Lysolecithin | 0.05 | 0.10 |
| | Bis-ethoxydiglycol cyclohexane 1,4- | 0.50 | 0.50 |
| | dicarboxylate | | |
| | Niacinamide | 4.00 | 4.00 |
| | Allantoin | 0.20 | 0.20 |
| | Adenosine | 0.04 | 0.04 |
| | Ammonium glycyrrhizate | 0.50 | 0.50 |
| Thickener | Diutan gum (Sphingomonas fermented extract) | 0.20 | 0.20 |
| | Ammonium polyacryloyldimethyl taurate | 0.10 | 0.10 |
| Oil phase | EMULFREE ® CBG MB(stearyl alcohol, | 1.00 | 1.00 |
| | butylene glycol cocoate, and ethyl cellulose) | | |
| | Caprylic/capric triglyceride | — | — |
| | Citron seed oil | 2.00 | 2.00 |
| | Dicaprylyl carbonate | 2.00 | 2.00 |
| | Bakuchiol | 0.50 | 0.50 |

### 3-2. Formulation Analysis of Examples 4 and 5 (Transdermal Permeability and Phase Separation Stability)

The content of the citron seed oil in Example 3 was lowered to 2.0% due to the strong ingredient odor thereof, and the content of lysolecithin, which may also increase the permeability of water-soluble ingredients, was increased from 0.02% to 0.05% to prepare an emulsifying composition of Example 4. As a result, the transdermal permeability of bakuchiol was slightly reduced, but the transdermal permeability of water-soluble active ingredients such as niacinamide and allantoin was increased.

Accordingly, the emulsifying composition of Example 5 was prepared by increasing the content of lysolecithin to 0.1%. In Example 5, no additional improvement in permeability was observed, but a higher permeability was observed compared to the compositions of Comparative Example 6 and Examples 2 and 3.

TABLE 6

| Formulation analysis | | Example 4 | Example 5 |
|---|---|---|---|
| Properties | Phase separation stability | Stable | Stable |
| | confirmed for 1 month at | | |
| | 50° C. | | |
| Transdermal | Niacinamide | 23.79 | 21.5 |
| penetration(ppm) | Allantoin | 1.8 | 1.19 |
| | Bakuchiol | 1.5 | 1.34 |

### Experimental Example 4. Preparation of Solubilizing Composition Using Active Ingredients (Niacinamide, Panthenol, Resveratrol)

### 4-1. Preparation of Solubilizing Compositions of Comparative Example 7 and Examples 6 to 8

The solubilizing compositions of Comparative Example 7 and Examples 6 to 8 were prepared according to the compositions set forth in Table 1. The ingredients constituting an aqueous phase in Table 1 were injected into a dissolving tank, heated (70 to 75° C.) and agitated to form the aqueous phase.

Subsequently, the ingredients constituting a solubilizing moiety 1 were injected into a separate dissolving tank and agitated at room temperature to form a solubilizing phase 1 and the ingredients constituting a solubilizing moiety 2 were injected into another dissolving tank and agitated at room temperature to form a solubilizing phase 2. The solubilizing moiety 1 (solubilizing phase 1) and the solubilizing moiety 2 (solubilizing phase 2) were added to the aqueous phase to prepare a soluble composition, which was allowed to cool to room temperature.

In addition, the ingredients constituting a pH control moiety were mixed in a separate dissolving tank and stirred at room temperature to prepare a pH control agent.

The solubilizing composition was mixed with niacinamide, panthenol, and resveratrol as thickeners and active ingredients (useful ingredients), followed by agitated to prepare a cosmetic composition containing useful ingredients.

TABLE 7

| | | Comparative | | | |
|---|---|---|---|---|---|
| Phase | Ingredient name | Example 7 | Example 6 | Example 7 | Example 8 |
| Aqueous | Purified water | to 100 | to 100 | to 100 | to 100 |
| phase | 2,3-Butanediol | 10.00 | 10.00 | 10.00 | 10.00 |
| | Bis-ethoxydiglycol cyclohexane | 0.50 | 0.50 | 0.50 | 0.50 |
| | 1,4-dicarboxylate | | | | |
| | Succinic acid | 0.50 | 0.50 | 0.50 | 0.50 |
| | Niacinamide | 2.00 | 2.00 | 2.00 | 2.00 |
| | Ammonium glycyrrhizate | 0.30 | 0.30 | 0.30 | 0.30 |
| | Panthenol | 0.20 | 0.20 | 0.20 | 0.20 |
| | Adenosine | 0.04 | 0.04 | 0.04 | 0.04 |
| Thickener | Diutan gum (Sphingomonas | 0.05 | 0.05 | 0.05 | 0.05 |
| | fermented extract) | | | | |
| Solubilizing | Dipropylene glycol | 5.00 | 5.00 | 5.00 | 5.00 |
| phase 1 | Resveratrol | 0.05 | 0.05 | 0.05 | 0.05 |
| | PEG-40 hydrogenated castor oil | 0.30 | — | | |
| | Glycereth-25 PCA isostearate | — | 0.30 | 0.60 | 0.90 |
| Solubilizing | Pentylene glycol | 3.00 | 3.00 | 3.00 | 3.00 |
| phase 2 | Lysolecithin | 0.02 | 0.02 | 0.02 | 0.02 |
| | PEG-40 hydrogenated castor oil | 0.20 | — | — | — |
| | Glycereth-25 PCA isostearate | — | 0.20 | 0.20 | 0.20 |
| | Flavor | 0.04 | 0.04 | 0.04 | 0.04 |
| pH control | Purified water | 5.00 | 5.00 | 5.00 | 5.00 |
| agent | Tromethamine | 0.40 | 0.40 | 0.40 | 0.40 |

### 4-2. Analysis of Transdermal Permeability of Comparative Example 7 and Examples 6 to 8

The formulation of the solubilizing composition was obtained using only diutan gum, which was found to have high transdermal permeability. A variation experiment for a solubilizing agent capable of stabilizing resveratrol, which is not readily dissolved in water, was conducted in advance, and PEG-40 hydrogenated castor oil and glycereth-25 PCA isostearate were selected as optimal resveratrol solubilizing agents.

In order to compare the transdermal permeability of the solubilizing compositions to which the two solubilizing agents were applied, a transdermal permeability test was performed on the active ingredients in the solubilizing compositions of Comparative Example 7 and Example 6. As a result, the transdermal permeability promotion effect of Example 6, in which 0.5% of glycereth-25 PCA isostearate was used, was significantly higher than that of Comparative Example 7 in which 0.5% of PEG-40 hydrogenated castor oil was used.

Then, the solubilizing compositions of Examples 7 and 8 were prepared by increasing the content of glycereth-25 PCA isostearate to 0.8% and 1.1%, respectively, and the transdermal permeability test was conducted in the same manner. As a result, the permeation improvement effect of niacinamide and panthenol was found in Example 7, where glycereth-25 PCA isostearate 0.8% was increased compared to 0.5%. Overall, Example 7, in which 0.8% of glycereth-25 PCA isostearate was used, and Example 8, in which 1.1% of glycereth-25 PCA isostearate was used, exhibited similar permeability of active ingredient.

TABLE 8

| Transdermal | Comparative | Example | Example | Example |
|---|---|---|---|---|
| penetration (ppm) | Example 7 | 6 | 7 | 8 |
| Niacinamide | 8.76 | 15.7 | 20.18 | 21.37 |
| Panthenol | 0.11 | 0.28 | 0.45 | 0.45 |
| Resveratrol | 0.23 | 0.35 | 0.36 | 0.36 |

### Experimental Example 5. Preparation of Solubilizing Composition Using Active Ingredients (Niacinamide, Ectoine, Resveratrol)

### 5-1. Preparation of Solubilizing Compositions of Examples 9 to 12

The solubilizing compositions of Examples 9 to 12 were prepared according to the compositions as shown in Table 9 in the same manner as in the solubilizing compositions of Comparative Example 7 and Examples 6 to 8, except that only the active ingredients were changed.

TABLE 9

| Phase | Ingredient name | Example 9 | Example 10 | Example 11 | Example 12 |
|---|---|---|---|---|---|
| Aqueous | Purified water | to 100 | to 100 | to 100 | to 100 |
| phase | Glycerin | 10.00 | 10.00 | 10.00 | 10.00 |
| | Bis-ethoxydiglycol | 0.50 | 0.50 | 0.50 | 0.50 |
| | cyclohexane 1,4- | | | | |
| | dicarboxylate | | | | |
| | 1,2-Hexanediol | 2.00 | 2.00 | 2.00 | 2.00 |
| | Niacinamide | 2.00 | 2.00 | 2.00 | 2.00 |
| | Ectoine | 1.00 | 1.00 | 1.00 | 1.00 |
| | Adenosine | 0.04 | 0.04 | 0.04 | 0.04 |
| Thickener | Diutan gum | 0.10 | — | — | — |
| | Xanthan gum | — | 0.10 | — | 0.10 |
| | Carbomer | — | — | 0.10 | 0.10 |
| Solubilizing | Dipropylene glycol | 7.00 | 7.00 | 7.00 | 7.00 |
| phase 1 | Glycereth-25 PCA | 0.50 | 0.50 | 0.50 | 0.50 |
| | isostearate | | | | |
| | Resveratrol | 0.05 | 0.05 | 0.05 | 0.05 |
| Solubilizing | 2,3-butanediol | 2.00 | 2.00 | 2.00 | 2.00 |
| phase 2 | Glycereth-25 PCA | 0.30 | 0.30 | 0.30 | 0.30 |
| | isostearate | | | | |
| | Lysolecithin | 0.02 | 0.02 | 0.02 | 0.02 |
| | Flavor | 0.04 | 0.04 | 0.04 | 0.04 |
| pH control | Purified water | — | — | 2.00 | 2.00 |
| agent | Tromethamine | — | — | 0.07 | 0.07 |

5-2. Analysis of transdermal permeability of Examples 9 to 12

In the previous experiment, diutan gum, which had almost no or a slightly negative effect on the transdermal permeability of the active ingredient, had a turbid appearance rather than being transparent in the aqueous solution. Therefore, it was necessary to examine other thickeners in order to prepare a transparent liquid composition. In this example, carbomer and xanthan gum were used as thickeners. The carbomer was neutralized with tromethamine before use.

In the solubilizing compositions of Examples 9, 10, and 11, which were prepared using the same amount (0.1%) of diutan gum, xanthan gum, and carbomer, there was a difference in the transdermal permeability of the active ingredients (niacinamide, ectoine, resveratrol), but there was no significant difference from diutan gum. In addition, Example 12, which used a combination of xanthan gum and carbomer, exhibited similar transdermal permeability of the active ingredients to that of Example 9, which used diutan gum alone. Therefore, the results of formulation analysis of Examples 9 to 12 showed that xanthan gum and carbomer do not interfere with the transdermal permeability of the active ingredients and thus may be used as thickeners suitable for transparent cosmetic compositions.

TABLE 10

| Transdermal | | | | |
|---|---|---|---|---|
| penetration (ppm) | Example 9 | Example 10 | Example 11 | Example 12 |
| Niacinamide | 15.70 | 20.18 | 18.43 | 15.10 |
| Panthenol | 2.18 | 3.37 | 1.91 | 2.34 |
| Resveratrol | 0.41 | 0.65 | 0.33 | 0.46 |

### Experimental Example 6. Preparation of Emulsifying Composition Using Active Ingredients (Niacinamide, Alpha-Bisabolol, Glabridin)

### 6-1. Preparation of Emulsifying Compositions of Comparative Examples 8 to 10 and Examples 13 and 14

The emulsifying compositions of Comparative Examples 8 to 10 and Examples 13 and 14 were prepared using the compositions set forth in Table 11 in the same manner as in Comparative Examples 1 to 3, except that only the active ingredients were changed.

TABLE 11

| | | Comparative | Comparative | Example | Example | Comparative |
|---|---|---|---|---|---|---|
| Phase | Ingredient name | Example 8 | Example 9 | 13 | 14 | Example 10 |
| Aqueous | Purified water | to 100 | to 100 | to 100 | to 100 | to 100 |
| phase | 2,3-Butanediol | 9.00 | 9.00 | 9.00 | 9.00 | 9.00 |
| | 1,2-hexanediol | 2.00 | 2.00 | 2.00 | 2.00 | 2.00 |
| | Bis-ethoxydiglycol cyclohexane | 0.50 | 0.50 | 0.50 | 1.00 | 2.00 |
| | 1,4-dicarboxylate | | | | | |
| | Glycereth-25 PCA isostearate | 0.50 | 0.50 | 0.50 | 0.50 | 0.50 |
| | Lysolecithin | 0.02 | 0.02 | 0.02 | 0.02 | 0.02 |
| | Niacinamide | 2.00 | 2.00 | 2.00 | 2.00 | 2.00 |
| | Adenosine | 0.04 | 0.04 | 0.04 | 0.04 | 0.04 |
| | Allantoin | 0.10 | 0.10 | 0.10 | 0.10 | 0.10 |
| Thickener | Carbomer | 0.10 | 0.10 | 0.10 | 0.10 | 0.10 |
| | Xanthan gum | 0.05 | 0.05 | 0.05 | 0.05 | 0.05 |
| | Ammonium polyacryloyldimethyl | — | 0.20 | — | — | — |
| | taurate | | | | | |
| | Acrylate/C10-30 alkyl acrylate | — | — | 0.20 | 0.20 | 0.20 |
| | crosspolymer | | | | | |
| Oil phase | TEGO ® Care 450(Polyglyceryl-3 | 1.00 | 1.00 | 1.00 | 1.00 | 1.00 |
| | methyl glucose distearate) | | | | | |
| | SENSANOV ™ WRWR(C20-22 | 0.50 | 0.50 | 0.50 | 0.50 | 0.50 |
| | Alkyl phosphate/C20-22 alcohol) | | | | | |
| | Citron seed oil | 1.50 | 1.50 | 1.50 | 1.50 | 1.50 |
| | Alpha-bisabolol | 0.50 | 0.50 | 0.50 | 0.50 | 0.50 |
| | Glabridin | 0.02 | 0.02 | 0.02 | 0.02 | 0.02 |
| pH | Purified water | 2.00 | 2.00 | 2.00 | 2.00 | 2.00 |
| control | Tromethamine | 0.10 | 0.10 | 0.24 | 0.24 | 0.24 |
| agent | | | | | | |

### 6-2. Formulation Analysis (Transdermal Permeability and Phase Separation Stability) of Comparative Examples 8 to 10 and Examples 13 and 14

O/W emulsifying compositions of Comparative Examples 8 to 10 and Examples 13 and 14 were prepared using TEGO® Care 450 (polyglyceryl-3 methyl glucose distearate) and SENSANOV™ WR (C20-22 alkyl phosphate/C20-22 alcohol) as emulsifiers. In the emulsifying composition of Comparative Example 8, the carbomer significantly lost its viscosity due to bis-ethoxydiglycol cyclohexane 1,4-dicarboxylate, resulting in phase separation. Xanthan gum is sticky and elastic, and thus is difficult to be used in a high content, and diutan gum has a unique slippery feeling when used.

Since the emulsifying composition was developed for care for oily skin prone to trouble, it needs to be absorbed with refreshing feeling. Therefore, in addition to small amounts of carbomer and xanthan gum, a thickener that was not sticky and had an emulsifying power without causing phase separation of the emulsion was required. Accordingly, Comparative Example in 9, which ammonium polyacryloyldimethyl taurate was used as a thickener in addition to carbomer and xanthan gum, Examples 13 and 14, and Comparative Example 10 in which acrylate/C10-30 alkyl acrylate crosspolymer was used as a thickener, were prepared. In the formulation in which acrylate/C10-30 alkyl acrylate crosspolymer was used, tromethamine was further added as a neutralizer and used at 0.24%.

In Comparative Example 9, which used 0.2% of ammonium polyacryloyldimethyl taurate as a thickener in addition to carbomer and xanthan gum, there was no phase separation, but the transdermal permeability of the active ingredient was significantly reduced. In Example 13, in which 0.2% of acrylate/C10-30 alkyl acrylate crosspolymer was used in addition to carbomer and xanthan gum, good transdermal permeability of the active ingredient was observed.

In addition, the emulsifying composition prepared by increasing the amount of bis-ethoxydiglycol cyclohexane 1,4-dicarboxylate from 0.5% to 1% so as to further improve the transdermal permeability of Example 13, maintained a stable phase and further exhibited enhanced transdermal permeability. However, the emulsifying composition of Comparative Example 10, which was the same as the emulsifying composition of Example 14, except that the amount of bis-ethoxydiglycol cyclohexane 1,4-dicarboxylate was increased to 2%, caused phase separation. Therefore, the emulsifying composition of Example 14 exhibited the best transdermal permeability of niacinamide, alpha-bisabolol, and glabridin, and formulation stability.

TABLE 12

| | | Comparative | Comparative | Example | Example | Comparative |
|---|---|---|---|---|---|---|
| Formulation analysis | | Example 8 | Example 9 | 13 | 14 | Example 10 |
| Properties | Phase separation | Phase | Stable | Stable | Stable | Phase |
| | stability confirmed | separation | | | | separation |
| | for 1 month at 50° C. | | | | | |
| Transdermal | Niacinamide | — | 0.93 | 19.23 | 21.43 | — |
| penetration | Alpha-bisabolol | — | 1.74 | 3.03 | 11.82 | — |
| (ppm) | Glabridin | — | 0.05 | 0.34 | 0.39 | — |

It will be apparent to those skilled in the art that various modifications and variations may be made in the disclosure without departing from the spirit or scope of the disclosure. Thus, it is intended that the disclosure cover such modifications and variations thereof, provided they fall within the scope of the appended claims and their equivalents.

1. A cosmetic composition for enhancing skin absorption of an active ingredient and formulation stability, the cosmetic composition comprising at least three ingredients selected from the group consisting of bis-ethoxydiglycol cyclohexane 1,4-dicarboxylate, lysolecithin, citron seed oil, and glycereth-25 PCA isostearate.

2. The cosmetic composition according to claim 1, wherein the active ingredient is selected from the group consisting of niacinamide, allantoin, bakuchiol, panthenol, resveratrol, ectoine, alpha-bisabolol, glabridin, rucinol, and tocopherol.

3. The cosmetic composition according to claim 1, wherein the cosmetic composition comprises bis-ethoxydiglycol cyclohexane 1,4-dicarboxylate, lysolecithin and citron seed oil.

4. The cosmetic composition according to claim 3, wherein the cosmetic composition comprises 0.3 to 2.0% (w/w) of the bis-ethoxydiglycol cyclohexane 1,4-dicarboxylate, 0.01 to 0.15% (w/w) of the lysolecithin, and 0.5 to 7.0% (w/w) of the citron seed oil.

5. The cosmetic composition according to claim 3, wherein the active ingredient comprises at least one selected from the group consisting of niacinamide, allantoin, alpha-bisabolol and bakuchiol.

6. The cosmetic composition according to claim 5, further comprising diutan gum and ammonium polyacryloyldimethyl taurate as thickeners.

7. The cosmetic composition according to claim 1, wherein the cosmetic composition comprises bis-ethoxydiglycol cyclohexane 1,4-dicarboxylate, lysolecithin, and glycereth-25 PCA isostearate.

8. The cosmetic composition according to claim 7, wherein the cosmetic composition comprises 0.3 to 2.0% (w/w) of the bis-ethoxydiglycol cyclohexane 1,4-dicarboxylate, 0.01 to 1.0% (w/w) of the lysolecithin, and 0.1 to 2.0% (w/w) of the glycereth-25 PCA isostearate.

9. The cosmetic composition according to claim 7, wherein the active ingredient comprises at least one selected from the group consisting of niacinamide, panthenol, resveratrol, ectoine, glabridin, alpha-bisabolol, rucinol, and bakuchiol.

10. The cosmetic composition according to claim 9, comprising glycereth-25 PCA isostearate as a solubilizing ingredient.

11. The cosmetic composition according to claim 9, further comprising diutan gum, xanthan gum, carbomer, or a combination of xanthan gum and carbomer, as a thickener.

12. The cosmetic composition according to claim 1, wherein the cosmetic composition comprises bis-ethoxydiglycol cyclohexane 1,4-dicarboxylate, lysolecithin, citron seed oil, and glycereth-25 PCA isostearate.

13. The cosmetic composition according to claim 12, wherein the cosmetic composition comprises 0.3 to 2.0% (w/w) of the bis-ethoxydiglycol cyclohexane 1,4-dicarboxylate, 0.01 to 1.0% (w/w) of the lysolecithin, 0.5 to 5.0% (w/w) of the citron seed oil, and 0.1 to 2.0% (w/w) of the glycereth-25 PCA isostearate.

14. The cosmetic composition according to claim 12, wherein the active ingredient comprises at least one selected from the group consisting of niacinamide, alpha-bisabolol, glabridin, and ectoine.

15. The cosmetic composition according to claim 14, further comprising carbomer, xanthan gum and acrylate/C10-30 alkyl acrylate crosspolymer as thickeners.

16. The cosmetic composition according to claim 1, wherein the cosmetic composition is formulated into at least one selected from the group consisting of a toner, a serum, an essence, a lotion, a cream, a powder, a foundation, a pack, a patch, a mask sheet, a gel, an ointment, an oil, a spray, and an ampoule.

17. A method for enhancing skin absorption of an active ingredient and improving formulation stability, the method comprising:
applying to the skin of a subject:
(a) a cosmetic composition comprising at least three ingredients selected from the group consisting of bis-ethoxydiglycol cyclohexane 1,4-dicarboxylate, lysolecithin, citron seed oil, and glycereth-25 PCA isostearate; and
(b) the active ingredient, wherein the active ingredient is either included in the cosmetic composition or applied separately.

18. The method according to claim 17, wherein the active ingredient is selected from the group consisting of niacinamide, allantoin, bakuchiol, panthenol, resveratrol, ectoine, alpha-bisabolol, glabridin, rucinol, and tocopherol.

19. A method of using a cosmetic composition to enhance skin absorption of an active ingredient and improve formulation stability, the method comprising:
applying to the skin of a subject:
(a) a cosmetic composition comprising at least three ingredients selected from the group consisting of bis-ethoxydiglycol cyclohexane 1,4-dicarboxylate, lysolecithin, citron seed oil, and glycereth-25 PCA isostearate; and
(b) an active ingredient, wherein the active ingredient is either included in the cosmetic composition or applied separately.
